(19) United States
(12) Patent Application Publication
(10) Pub. No.: US 2006/0074279 A1
(43) Pub. Date: Apr. 6, 2006

(54) Interactive dieting and exercise system

(76) Inventor: Evgeny Brover, Herzlia (IL)

Correspondence Address: MARTIN MOYNIHAN;c/o ANTHONY CASTORINA, SUITE 207, 2001 JEFFERSON DAVIS HIGHWAY, ARLINGTON, VA, 22202, US

(21) Appl. No.: 10/951,779

(22) Filed: Sep. 29, 2004

(51) Int. Cl. A61B 5/00

(52) U.S. Cl. 600/300

(57) ABSTRACT

A system for providing a user with a diet and exercise program and on-line support, comprises: a central server having a diet and exercise model, a database of personal profiles of a plurality of users, a communication unit for communicating with said plurality of users over a network, an advice calculator unit for using said diet and exercise model together with respective personal profiles to plan a personalized diet and exercise program for a given user, and a scheduler for scheduling sending of details of said program to said remote user via said communication unit, thereby to provide said user with a time-based diet and exercise program. Additionally, a query unit provides a facility for allowing the user to make on-line queries of the database. Queries may range from asking about nutritional values of given foods to requesting modifications of the diet and exercise program.

### FIELD AND BACKGROUND OF THE INVENTION

The present invention relates to an interactive dieting and exercise system and, more particularly, but not exclusively to an interactive system that provides users with dieting and exercise programs and responds to user queries in a way that integrates dieting and exercise.

Generally, users are able to understand that if they eat less they will gain less weight and may even lose weight. Users have difficulty, however in setting themselves definite health targets and ways of achieving those targets without professional advice. The professional adviser, however, is not likely to be with them at the crucial moment of a meal when they are offered an extra piece of cake, and neither is the professional adviser likely to explain to them how to translate an hour of moderate exercise into additional calories that may be eaten. Furthermore, since professional advice is expensive, the user is not likely to ask the professional adviser for regular updates of daily menus and exercise programs. Nevertheless an important factor in the success or otherwise of a dieting program is persistence on the part of a user. It is important that the user incorporates daily changes in circumstances into the program rather than ignore the program.

Dieting systems are available over the Internet, and can provide the user with much nutritional information, dieting and exercise programs and even daily diet sheets personalized for the individual user according to information that the user provides. However the existing systems are unable to modify the advice in real time in the light of information provided subsequently by the user. Many users have a preferred system of exercises, some prefer swimming, others weights and others running, whilst others still might wish to mix and match. Furthermore, and more crucially, daily changes in circumstances, such as the gym being closed, or having to work late on a particular day, can upset the program. The user thus needs to incorporate these changes into the program rather than ignore them.

Again, users may have preferred tastes in food, yet existing systems provide menus that may be ignored by the user because the systems are unable to make automatic use of user reactions. The likelihood of a menu being ignored is a particularly serious issue in the dieting field where personal motivation of the user is highly important, as explained above. Generally, the psychological issue is most commonly approached using the group support method. But group meetings usually do not exceed once a week, which is not really sufficient.

Essentially a dieting and exercise program is a complex system in which calories input must be calculated for different types of food, calories burned must be calculated for different types of exercise, the two must be balanced and the results compared with a personal profile and made available to the user when seated at his kitchen table or in the restaurant or at the supermarket or in the gym without the cost of a constantly available professional adviser. Furthermore a diet is not simply a matter of adding up calories. A diet must be balanced in terms of food groups and nutrients and preferably should not give rise to undesirable physiological phenomena such as sharp changes in blood-sugar levels. Persons need to be able to take into account all of the above, especially in relation to circumstances that may change at short notice.

Existing dieting models such as that used in the Weightwatcher program are heavily simplified so that ordinary people can use them on a day to day basis, but the result is a system that fails to fulfill many of the above functions.

There is thus a widely recognized need for, and it would be highly advantageous to have, a dieting and exercise system devoid of the above limitations.

### SUMMARY OF THE INVENTION

According to one aspect of the present invention there is provided a system for diet and exercise comprising:

a central server having a diet and exercise model,

a database of personal profiles of a plurality of users,

a communication unit for communicating with said plurality of users over a network,

an advice calculator unit for using said diet and exercise model together with respective personal profiles to plan a personalized diet and exercise program for a given user,

an interaction unit for obtaining at least one query from said given user, using said query with said diet and exercise model and said corresponding personal profile to provide a modification to said personalized diet and exercise program, and sending said modified advice to said communication unit for communication to said remote user.

The system may comprise a scheduler for scheduling sending of details of said program to said remote user via said communication unit, thereby to provide said user with a time-based diet and exercise program.

Preferably, said scheduler is user reprogrammable via said communication unit to provide a customized user schedule.

Preferably, said communication unit is configured for communication via an IP-based network.

Preferably, said communication unit is configured for communication via web-based forms.

Preferably, said communication unit is configured for communication via text messaging, or via email or via webforms or by voicemail or by any other suitable method. In an embodiment the communication unit is able to send videos such as exercise training videos.

Preferably, said communication unit is also configured for SMS or MMS messaging via a cellular telephony network.

Preferably, said communication unit is also configured for communication via an Interactive TV network. Interactive TV may involve a dedicated TV channel or text or media messaging, or both.

Preferably, said communication unit is also configured for communication via the public switched telephone network.

Preferably, said dieting and exercise model comprises a points based system in which food is assigned points in a first polarity and exercise is assigned points in a second polarity, and said advice calculator totals food and exercise to a total determined from a respective personal profile.

Preferably, said total is determined from said personal profile according to predefined criteria in said model.

The system may comprise a plurality of daily menus for each one of a plurality of totals.

Preferably, there is provided at least fifty menus for each of said plurality of totals.

The system may comprise a learning engine for recognizing patterns from user interactions to modify said menus for a given user if the modified menus are made part of the personal profile. For that matter the modified menus can be placed into a general database and then be made available to all users.

Preferably, said dieting and exercise model comprises a points-based system in which different kinds and portion sizes of food are assigned points in a first polarity and different kinds, levels and durations of exercise are assigned points in a second, opposite polarity and said advice calculator totals food and exercise to a total determined from a respective personal profile.

Preferably, said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates at least one of a personal menu and a personal exercise program using said totals and said constraints.

The system may comprise a learning engine to recognize patterns from query information from a given user to modify said at least one of a personal menu and a personal exercise program.

Preferably, said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates both a personal menu and a personal exercise program using said totals and said constraints.

According to a second aspect of the present invention there is provided a system for diet and exercise comprising:

a central server having a diet and exercise model,

a database of personal profiles of a plurality of users,

a communication unit for communicating with said plurality of users over a network,

an advice calculator unit for using said diet and exercise model together with respective personal profiles to plan a personalized diet and exercise program for a given user, and

a scheduler for scheduling sending of details of said program to said remote user via said communication unit, thereby to provide said user with a time-based diet and exercise program.

Preferably, said scheduler is user reprogrammable via said communication unit to provide a customized user schedule.

Preferably, said communication unit is configured for communication via an IP-based network.

Preferably said communication unit is configured for communication via web-based forms.

Preferably, said communication unit is configured for communication via messaging.

Preferably, said communication unit is configured for SMS messaging via a cellular telephony network.

Preferably, said communication network is also configured for communicating via the public switched telephone network.

Preferably, said communication network is configured for communicating via an interactive television network.

Preferably, said dieting and exercise model comprises a points based system in which food is assigned points in a first polarity and exercise is assigned points in a second polarity, and said advice calculator totals food and exercise to a total determined from a respective personal profile.

Preferably, said total is determined from said personal profile according to predefined criteria in said model.

The system may comprise a plurality of daily menus for each one of a plurality of totals.

Preferably, there is provided at least fifty menus for each of said plurality of totals.

The system may comprise a learning engine for recognizing patterns from user interactions to modify said menus for a given user.

Preferably, said dieting and exercise model comprises a points-based system in which different kinds and portion sizes of food are assigned points in a first polarity and different kinds, levels and durations of exercise are assigned points in a second, opposite polarity and said advice calculator totals food and exercise to a total determined from a respective personal profile.

Preferably, said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates at least one of a personal menu and a personal exercise program using said totals and said constraints.

The system may comprise a learning engine configured to additionally use past query information from a given user to modify said at least one of a personal menu and a personal exercise program.

Preferably, said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates both a personal menu and a personal exercise program using said totals and said constraints.

According to a third aspect of the present invention there is provided an interactive system for diet and exercise comprising:

a user profile builder for obtaining information from a user and building a profile of data from said user relevant to diet and exercise;

a diet and exercise model for utilizing said profile to assign a given user with a score indicating nutritional need;

a database of nutritional plans associated with different scores;

a database of exercise plans;

a personalization unit for assigning a given user with a combined nutritional and exercise plan based on a corresponding score;

a scheduler for scheduling notifications to users of their respective plans;

a query unit for receiving queries from respective users and responding with requested data or with modifications to said plans; and

a learning unit for discerning patterns in queries of respective users and using said patterns to modify said nutritional plans to provide a set of customized nutritional plans for said respective users.

Preferably, said users are remotely connected thereto by an IP-based network.

Preferably, users are remotely connected thereto via a cellular telephony network.

Preferably, said users are also able to connect thereto via an interactive television network.

Preferably said users are also able to connect thereto via the public switched telephone network.

According to a fourth aspect of the present invention there is provided a method for interactive support for users for a diet and exercise program comprising:

providing an electronic diet and exercise model,

providing a database of personal profiles of a plurality of remote users,

providing a communication unit for communicating with said plurality of remote users over a network,

using said diet and exercise model together with respective personal profiles to plan a personalized diet and exercise program for a given remote user,

obtaining at least one query from said given remote user,

using said query with said diet and exercise model and said corresponding personal profile to provide a modification to said personalized diet and exercise program, and

sending said modified advice to said remote user.

According to a fifth aspect of the present invention there is provided a method for interactive support of users in a diet and exercise program comprising:

providing an electronic diet and exercise model,

constructing a database of personal profiles of a plurality of remote users,

communicating with said plurality of remote users over a network,

using said electronic diet and exercise model together with respective personal profiles to plan a personalized diet and exercise program for a given user, and

scheduling sending of details of said program to said remote user thereby to provide said user with a time-based diet and exercise program.

According to a sixth aspect of the present invention there is provided a method for interactive support of users in a diet and exercise program comprising:

obtaining information from a user and building a profile of data from said user relevant to diet and exercise;

utilizing said profile to assign a given user with a score indicating nutritional need;

providing a database of nutritional plans associated with different scores;

providing a database of exercise plans;

assigning a given user with a combined nutritional and exercise plan based on a corresponding score;

scheduling notifications to users of their respective plans;

receiving queries from respective users and responding with requested data or with modifications to said plans; and

discerning patterns in queries of respective users, and

using said patterns to modify said nutritional plans to provide a set of customized nutritional plans for said respective users.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified block diagram showing an Interactive Dieting and Exercise system for use by remote users via a network, according to a first preferred embodiment of the present invention;

FIG. 2 is a simplified block diagram showing an Interactive Scheduler for use in the embodiment of FIG. 1;

FIG. 3 is a simplified block diagram showing a query engine for SMS messaging for use in the embodiment of FIG. 1;

FIG. 4 is a simplified block diagram showing an alternative query engine for use in the embodiment of FIG. 1;

FIG. 5 is a simplified diagram illustrating a learning engine for use in the embodiment of FIG. 1; and

FIG. 6 is a simplified diagram illustrating a preferred model for use in the embodiment of FIG. 1.

### DESCRIPTION OF THE PREFERRED EMBODIMENTS

The present embodiments comprise an interactive system available to remote users over a network that makes an electronic dieting and exercise model available to the remote users. The system allows the model to interact in a personalized way with each remote user via a user profile and on-line queries. The interactions include those initiated by the system using a scheduler, which again can be personalized, and those initiated by the user, in which the user interrogates the model. User queries can be used as the basis for learning by the model to add to or otherwise change the user profile or datasets with which the model interacts via the user profile.

The principles and operation of an interactive system for dieting and exercise according to the present invention may be better understood with reference to the drawings and accompanying description.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

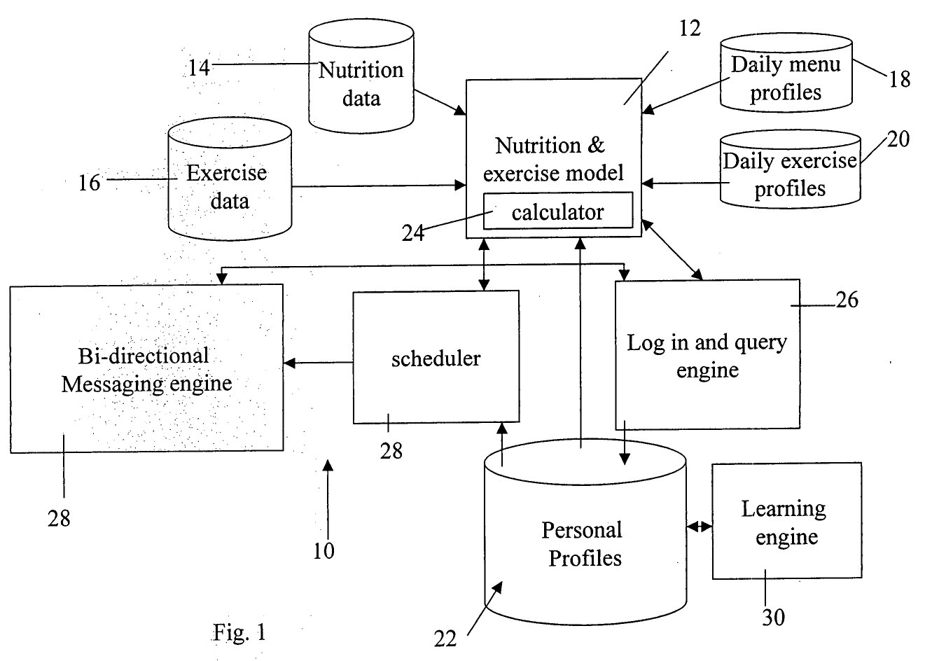

Reference is now made to FIG. 1, which is a simplified block diagram illustrating a generalized embodiment of the present invention. In FIG. 1, system 10, typically based on one or more servers connected to a network, comprises a series of engines and databases. One of the servers preferably supports an electronic nutrition, or diet, and exercise model 12. The model is supported by database 14 of nutrition information regarding different foods. The nutrition information may include data such as the food group or groups to which the food belongs, calorific value for given sized portions, quantity of vitamins, minerals and other dietary essentials, quantity of dietary undesirables such as bad cholesterol, and any other information of use in advising on nutrition. In particular, for a points-based nutrition system such as that used in the Weightwatchers system, the database may include the dietary points of the nutrition system.

In an embodiment database 14 may include material from well-known standard databases such as the regularly updated USDA nutritional database. In a preferred embodiment database 14 may include an online connection to the well-known standard database.

The model is also supported by exercise database 16. The database contains data of different types of exercise, recommended frequencies and durations for different user profiles, and information on how they may modify a dietary program. Thus carrying out an hour's exercise of a certain kind may allow a user of a given profile to eat an additional 100 calories or an additional point on the weightwatcher's system. In one embodiment the exercises are divided within the database into three categories, mild, moderate and vigorous, and each category carried out for a certain amount of time earns a user a given number of additional calories or points.

It is noted that the databases may contain not just textual information but pictures and video as well. Thus the exercise database may for example include instruction videos for given exercises, which can be made available to users.

In one embodiment the model simply defines the amount that a person can eat during the course of the day based on information about the user and then there is a database 18 of menus for each amount. Thus there may be 50 different menus for each amount, each menu giving balanced nutrition.

Depending on user feedback the system is able to send out warning messages based on upsets in the balance between food intake and exercise, or based on any other criterion as desired. For example, a user who reports not taking his exercise one day may be warned to reduce the quantity of consumption the following day.

In another embodiment the model simply defines the types of foods that can be eaten. For example the Atkins diet emphasizes proteins and fats but excludes carbohydrates. Again such a model can be supported by menus.

In a preferred embodiment the Interactive system provides a choice of dieting models or system. The user simply chooses his preferred model.

In an alternative embodiment, the model is not supported by actual menus but rather by menu profiles. Thus database 18 comprises daily (or for that matter weekly or monthly) nutrition profiles. The nutrition profile is an outline of how a person eats, or should eat if following a healthy nutrition and exercise program, during the course of the day. Thus the profile sets out a certain type of meal for eating early in the morning, with slots for different breakfast-type foods, a midday meal of a certain kind, an evening meal of a certain kind etc. For each meal the profile includes a slot for protein, a slot for green vegetables etc. The model can then be used to fill in the slots in the nutrition profile. The nutrition profiles themselves are generic, meaning they can be used in different ways for different kinds of users. However there may be several profiles, for different lifestyles of users. Thus some users may have a main meal at lunchtime and others in the evening. Nutritionists generally recommend eating a larger number of small meals rather than a small number of larger meals, however this is not always practical for all users, and therefore different profiles may be provided for the different numbers of meals that different users are able to eat. Furthermore, there are certain foods or food groups that are not recommended for daily eating, but which can be eaten once or twice a week, and different profiles can be used to incorporate such foods. Thus some users may have lifestyles that include a special meal at the weekend in which such foods may be incorporated. Other users may have lifestyles that involve going out one evening a week, and such foods may be incorporated in this way. As will be explained below, such issues may also be incorporated via the machine learning facility provided with the system.

Additionally there is provided a database 20 of daily (or weekly or monthly) exercise profiles. Different profiles define different exercise patterns to suit different lifestyles, and the model is able to fit into slots in the profile, suitable exercises for the given user.

The model is further provided with a database 22 of personal profiles for different users of the system. The personal profiles are initially obtained from the users as they log into the system and contain information such as age, sex, height, weight, level of fitness, lifestyle information and the like. The personal profile can also contain medical information about the user, such as the results of recent blood tests, whether the user is allergic to anything, whether the user suffers from diabetes or anything else that affects the diet, and these can be used to modify the diet presented to the user. Thus the system can observe that the user lacks say vitamin B or iron and modify the diet accordingly.

Likewise personal preferences or other dietary requirements can be stored in the database.

The model works through a calculator unit 24 which uses data from the nutrition and exercise databases according to the equations provided in the electronic model to fill in the slots in the diet and exercise profiles according to constraints derived from the personal profile. The result is a personalized diet and exercise program for a given user.

The system further comprises a log-in and query unit 26, essentially an interaction unit, which obtains queries from individual users, understands the queries and uses the model and all the associated data to provide the user with the required information. The required information may be nutritional data about a particular food, or it may be information about a given exercise. Alternatively the query may be somewhat more complex, requiring a modification to the user's diet and exercise program. The user may ask for the rest of his daily menu to be modified due to the fact that he is now eating a steak, or due to the fact that fruit is currently unavailable. The unit 26 also allows users to log in so that their personal profiles can be identified in order to answer the query. In certain cases, log-in may be transparent to the user, for example if the user is contacting the system via SMS from his mobile telephone then the system should be able to log him in automatically.

In one embodiment the user may have for example an allowance for a given day of 18 points, but he has already consumed 12 points. Now he asks whether he can have a steak, the steak being worth 8 points. Since he does not have enough points remaining the system replies in the negative.

The system preferably further comprises a scheduler 28, which schedules the sending of data regarding the daily exercise and nutrition program to the user. The scheduler typically works by initiating an automatic query of the model for a given user at a given time, and sends the result to the user. Thus the scheduler may ask for a daily program for the user at the beginning of each day and send it to the user. Such a daily system is suitable for e-mail but SMS is limited to between sixty and seventy characters for a message. Thus for SMS-based users, the scheduler would typically query the system several times a day, say once in advance for each meal and once for the exercise session. Alternatively it may send double or triple or even larger SMS messages. The scheduling is typically initially defined by the diet and exercise profiles or from information obtained from the user at registration, but may be modified by the user as time goes on. Thus a user who does his shopping once a week may want an outline of the next week's nutrition program at the time (s)he does his shopping. Other users may want their nutrition information a day in advance and others may be satisfied with being told in a single message sent at the start of each day.

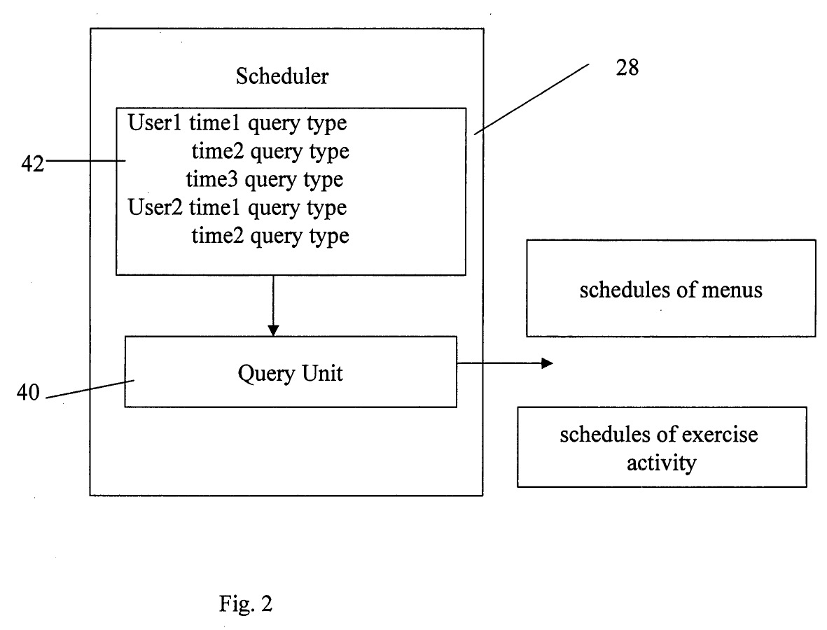

Thus the scheduler selects a given user and a time, queries the database, and sends the query results to the remote user so that the user obtains a time-based diet and exercise program. A preferred embodiment of the scheduler is shown in FIG. 2, in which it is shown to comprise a query unit 40 that formulates queries for the model. The queries are set in accordance with a scheduling database 42 that lists users and, with each user a time schedule with types of queries. Thus for a given user there may be a once a week query for a general outline of next week's food needs on shopping day, a daily menu provided each evening for the next day and a daily exercise schedule provided at 6 pm for the user's 7 pm gym session. The query unit is told by the database to formulate the relevant queries and provides the results as messages to the user.

The system further includes a communication unit, for example bidirectional messaging unit 30, which manages communications with remote users over a network such as the cellular telephony network, the cable TV network or the Internet. In addition the system can make use of the public switched telephone network (PSTN) via such protocols as DECT and SS7. Information from the system is packaged into messages in the media being used, for example e-mail or short text messages and sent to the user. Likewise information received from the user is sent inwards to the appropriate part of the system. The information that is received, whether from direct user input or from sensor devices, can then be used together with timing information in decision making.

The communication unit is configured for communication via one or more of an IP-based network, a cellular network, a cable or other TV network that has an interactive element, and any other network that may be available at the time and uses communication media over the given network as appropriate, such as text messaging over the Internet or the Cellular Networks, or text messaging using DECT over the PSTN, or e-mail over the Internet, web-forms over the Internet and the like. Likewise voice messages and voice recognition may be used.

It is noted that for the cable (or satellite) TV network, information can be provided via a dedicated channel, typically an Interactive channel, to which the user may be asked to subscribe. Alternatively or additionally the TV messaging system could be used.

There is preferably additionally provided a learning engine 30, which allows personal profiles to be modified on the basis of information gathered about the given user. The learning engine is preferably programmed to spot certain patterns, such as a user often asking about a given food, or about a user being particularly concerned about modifying the diet for a given day, and the like, and to use the information in the profile. Thus the nutrition program is over time able to take into account such features as the user particularly liking a certain style of food etc.

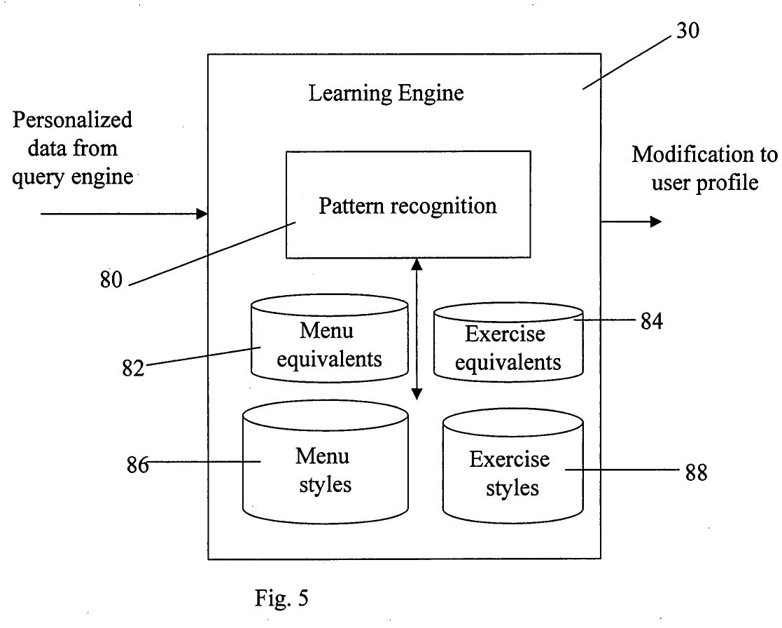

The learning engine is described in greater detail below with respect to FIG. 5.

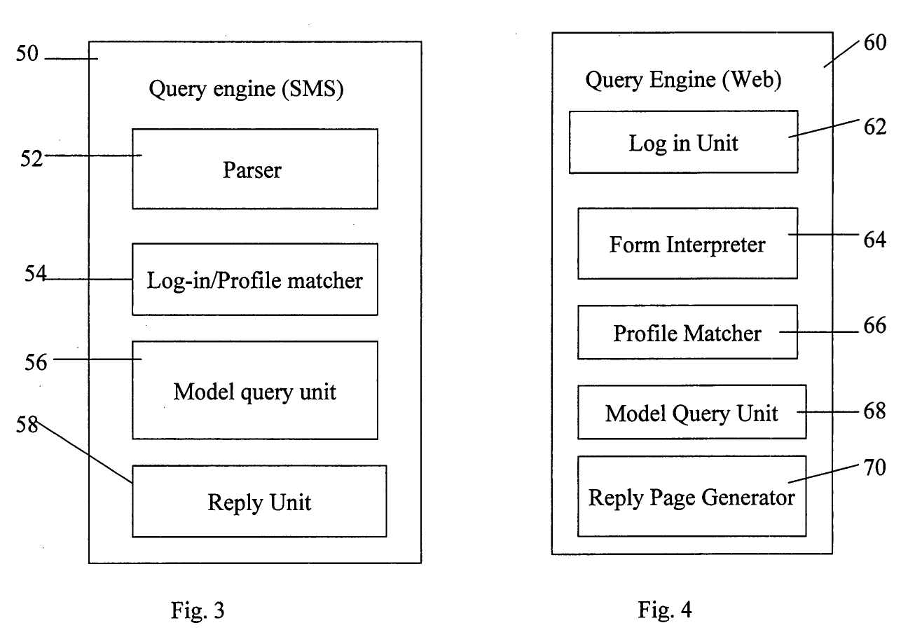

Reference is now made to FIG. 3, which shows one preferred embodiment of the Interactive log-in and query unit 26. The unit 50 shown in FIG. 3 is for queries via text messaging, for example SMS messages from a mobile telephone. The query is received and parsed, by parser 52, so that the message can be understood. Typically the central theme of a message on a diet and exercise system is going to be a kind of food or a kind of exercise. Thus parsing involves the two stages of firstly identifying this central theme and secondly identifying what kind of question is being asked about the central theme.

In one embodiment messages are about either food or exercise. A first component of the message is a digit of a four part code, say for example the digits 1 to 4, where 1 means yes, 2 means no, 3 means later and 4 means change. The subject of the message follows in the form of the name of a food or of an exercise. The skilled person will appreciate that the parsing of such a message is relatively trivial.

A log-in/personal profile finder unit 54 logs the user in so that the user can be identified and the appropriate personal profile be retrieved. Similarly any recent advice sent to the given user can be retrieved. Certain kinds of query may not require logging and finding the personal profile. For example straightforward queries about the nutritional makeup of a given food or the points available for a given exercise, can be answered directly from information stored in the nutrition or exercise database without requiring the user to identify himself. If the user's identity is required then it can be obtained from the caller line identification (CLI) accompanying the text message and this can be used for a log-in process that is transparent to the user, in the sense that the user is not aware of it. A model query unit 56 then formulates a query which can be sent to the model and the result is sent to reply unit 58 which formulates the model reply into a message that can be understood by a human. The message is then sent to the user.

Reference is now made to FIG. 4, which is a simplified diagram illustrating a modified interactive query unit 60 for using with queries obtained over the web via a web form. The user accesses a website and enters his or her query on a query form provided on the website. The form is structured so that meanings are readily apparent to the electronic system and parsing becomes a relatively trivial issue, however the user generally has to actively log in, since the web cannot be relied upon to identify the user. Thus unit 60 has a log in unit 62 for accepting log in information such as a user name and password and identifying the user. A form interpreter 64 carries out the same task as the parser of FIG. 3. A profile matcher 66 retrieves the user profile if needed to help answer the question. A query formulator 68 formulates a query which can be sent to the model and a page generator 70 generates a reply page which presents the reply as a web page to the user.

The skilled person will understand how to combine the query engine with a voice recognition system to provide a system that recognizes and understand queries left by voice.

In a preferred system numerous different types of communication are allowed. Preferably individual users are allowed to connect to the system via any kind of client device that they happen to have at hand. Thus a message sorter determines what kind of message is being received and sends it to the appropriate query device.

Reference is now made to FIG. 5, which is a simplified diagram illustrating a preferred embodiment of the learning engine 30. The purpose of the learning engine is to make use of queries from a given user, identify patterns, and incorporate those patterns into the user profile so that the user can be provided with more appropriate advice. The learning engine comprises a pattern recognition unit 80 which may make use of any known pattern identification system, for example neural networks, rule-based identification and the like. The pattern recognizer may make use of a nutritional database 82 showing which foods are substitutes for which other foods, and a database 84 which groups foods into food styles. For example it may become clear that a given user likes Mediterranean style food, whereas another likes Indian food etc. Another may be a vegetarian and yet another may prefer a North European diet. Having these eating styles available to the pattern matcher allows the system to see patterns in the choice of food of the user that the user himself may not even be aware of. Likewise the pattern matcher may be associated with a database 86 of exercise equivalents and a database 88 of exercise styles or patterns, with which it can perform a similar exercise.

In the embodiment referred to above in which fixed predetermined menus are used, the learning unit may use previous queries to directly modify the menus for the given user.

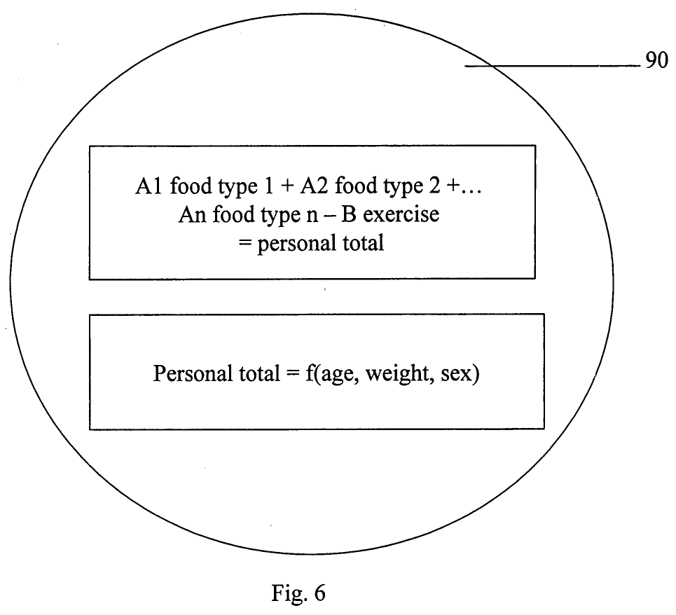

Reference is now made to FIG. 6, which illustrates a simplified dieting and exercise model 90. The particular model shown is compatible with the model used in the weightwatchers system although it will be appreciated that other, models can be used instead. The weightwatchers model essentially assigns a foodstuff a point value based on the number of calories. The point value is then increased if the food has undesirable substances therein such as cholesterol and decreased if it has desirable nutritional substances. Essentially a point is given for every approximately every 100 calories and then the total is increased or decreased depending on the nutritional value of the food. Green vegetables which are highly nutritional but have low calorific value are zero rated, whereas red meat or fried potato and the like are very highly rated. The user is then given a points total for daily consumption based on such variables as weight, height, sex, etc. Thus a 24 year old woman of a given height and weight for example may be given a daily allowance of 18 points.

The model further allows points to be deducted for exercise. Exercise is divided, as explained above into levels, light, moderate and intense, and then preselected durations are given a different number of points. The points earned in exercise can be deducted from the total, so that in effect the user is able to eat more in view of the exercise taken.

Typically the model provides additional constraints. The user is required to eat a certain minimal amount of different food types, for example a certain minimal amount of protein, a minimal amount of green vegetables etc. In addition there may be a maximum number of points that the user is allowed to deduct as a result of exercise. Furthermore the model may be constrained not to permit intense exercise to say a user of a given weight or a given age without a buildup of light and moderate exercise in advance.

In a preferred embodiment the system may take into account data gathered from the user, for example via medical sensors. Thus the user may have a watch that can take his pulse, or a sensor that can pick up an ECG signal. Other devices may estimate calories being burnt on the basis of different kinds of information. An intelligent toilet system can analyze a user's waste products. The outputs of any of these devices can be used as inputs to the system to be taken into account by the model.

The messages sent to the user may contain hints and tips as well as information about the actual nutrition and exercise program. Furthermore the messages may contain commercial advertising information and the information may be customized for the user. For example the cellular system can make location information available so that the system can provide relevant information to the user about local restaurants or shops. Likewise the system can be linked to a particular shop or chain of shops so that along with the daily or weekly menu, appropriate products from the shop can be recommended.

In one preferred embodiment the system is provided with specialist ringtones. The source of messages on the system is identified by the user's phone and a particular ringtone is used which is different from the ringtones of the regular telephone calls or messages. Such a ringtone may be downloaded on signing up with the system, and it is seen as particularly advantageous to use such a special ringtone as the user learns to recognize it and associate it instinctively with food or exercise.

Preferably the system provides the users with progress reports. Feedback from the users may be gathered from their messages to the system, or from the users filling out forms at regular intervals or from sensors or like devices whose outputs are used with the system. The reports may tell the user about their progress, recommend changes to the program and the like. Likewise it is possible to update a user's personal profile in accordance with his progress.

As a further point it is noted that if the client device of the user is his mobile telephone, it is possible for the system to determine in which time zone the user is located and modify the timing of messages in accordance therewith.

As a further point, the system need not be solely model-based. It is also possible for the system to have a point of access for professionals and organizations, for example a hospital, a clinic or the like or for direct use by for example a personal fitness trainer. Any of these may use the system as a way of providing information and supplementing his services. The system also allows the trainer to manage his client database and further allows him to access the progress reports as discussed above so that he can keep himself updated regarding his individual clients. From the financial point of view the system may also provide a trainer with the ability to manage the personal accounts of his clients.

An advantage of the present embodiments to the cellular telephone companies and other organizations that provide text messaging is that they introduce new people to the world of text messaging and increase the amount of messaging to people who are already familiar therewith. The same applies to introducing users to MMS and video by cellular device and the like.

A further advantage of the present system is that since it is automatic it can function in several languages, as opposed to the personal trainer himself, who is often not so versatile. The system may use the personal profile to ask a user to identify a language he would like to use.

It is expected that during the life of this patent many relevant interactive devices and systems for use by remote users over a network will be developed and the scope of the corresponding terms herein is intended to include all such new technologies a priori.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A system for diet and exercise comprising:
a central server having a diet and exercise model,
a database of personal profiles of a plurality of users,
a communication unit for communicating with said plurality of users over a network,
an advice calculator unit for using said diet and exercise model together with respective personal profiles to plan a personalized diet and exercise program for a given user,
an interaction unit for obtaining at least one query from said given user, using said query with said diet and exercise model and said corresponding personal profile to provide a modification to said personalized diet and exercise program, and sending said modified advice to said communication unit for communication to said remote user.

2. The system of claim 1, further comprising a scheduler for scheduling sending of details of said program to said remote user via said communication unit, thereby to provide said user with a time-based diet and exercise program.

3. The system of claim 1, configured to operate in conjunction with at least one sensor based system at a user to provide a diet and exercise program modified in light of output data of said sensor-based system.

4. The system of claim 2, wherein said scheduler is user reprogrammable via said communication unit to provide a customized user schedule.

5. The system of claim 1, wherein said communication unit is configured for communication via an IP-based network.

6. The system of claim 5, wherein said communication unit is configured for communication via web-based forms.

7. The system of claim 1, wherein said communication unit is configured for communication via at least one of a group comprising text messaging, voice mail, a voice interactive system and multi-media messaging (MMS).

8. The system of claim 7, wherein said communication unit is configured for SMS messaging via a cellular telephony network.

9. The system of claim 1, wherein said communication unit is configured for communication via an Interactive TV network.

10. The system of claim 1, wherein said communication unit is configured for communication via the public switched telephone network.

11. The system of claim 1, wherein said dieting and exercise model comprises a points based system in which food is assigned points in a first polarity and exercise is assigned points in a second polarity, and said advice calculator totals food and exercise to a total determined from a respective personal profile.

12. The system of claim 11, wherein said total is determined from said personal profile according to predefined criteria in said model.

13. The system of claim 12, comprising a plurality of daily menus for each one of a plurality of totals.

14. The system of claim 13, wherein there is provided at least fifty menus for each of said plurality of totals.

15. The system of claim 13, further comprising a learning engine for recognizing patterns from user interactions to modify said menus for a given user.

16. The system of claim 1, wherein said dieting and exercise model comprises a points-based system in which different kinds and portion sizes of food are assigned points in a first polarity and different kinds, levels and durations of exercise are assigned points in a second, opposite polarity and said advice calculator totals food and exercise to a total determined from a respective personal profile.

17. The system of claim 1, wherein said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates at least one of a personal menu and a personal exercise program using said totals and said constraints.

18. The system of claim 17, further comprising a learning engine to recognize patterns from query information from a given user to modify said at least one of a personal menu and a personal exercise program.

19. The system of claim 1, wherein said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates both a personal menu and a personal exercise program using said totals and said constraints.

20. A system for diet and exercise comprising:
a central server having a diet and exercise model,
a database of personal profiles of a plurality of users,
a communication unit for communicating with said plurality of users over a network,
an advice calculator unit for using said diet and exercise model together with respective personal profiles to plan a personalized diet and exercise program for a given user, and
a scheduler for scheduling sending of details of said program to said remote user via said communication unit, thereby to provide said user with a time-based diet and exercise program.

21. The system of claim 20, wherein said scheduler is user reprogrammable via said communication unit to provide a customized user schedule.

22. The system of claim 20, wherein said communication unit is configured for communication via an IP-based network.

23. The system of claim 22, wherein said communication unit is configured for communication via web-based forms.

24. The system of claim 20, wherein said communication unit is configured for communication via messaging.

25. The system of claim 24, wherein said communication unit is configured for for at least one of a group comprising text messaging, voice mail, a voice interactive system and multi-media messaging (MMS) via a cellular telephony network.

26. The system of claim 20, wherein said communication network is configured for communicating via the public switched telephone network.

27. The system of claim 20, wherein said communication network is configured for communicating via an interactive television network.

28. The system of claim 20, wherein said dieting and exercise model comprises a points based system in which food is assigned points in a first polarity and exercise is assigned points in a second polarity, and said advice calculator totals food and exercise to a total determined from a respective personal profile.

29. The system of claim 28, wherein said total is determined from said personal profile according to predefined criteria in said model.

30. The system of claim 29, comprising a plurality of daily menus for each one of a plurality of totals.

31. The system of claim 30, wherein there is provided at least fifty menus for each of said plurality of totals.

32. The system of claim 30, further comprising a learning engine for recognizing patterns from user interactions to modify said menus for a given user.

33. The system of claim 20, wherein said dieting and exercise model comprises a points-based system in which different kinds and portion sizes of food are assigned points in a first polarity and different kinds, levels and durations of exercise are assigned points in a second, opposite polarity and said advice calculator totals food and exercise to a total determined from a respective personal profile.

34. The system of claim 20, wherein said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates at least one of a personal menu and a personal exercise program using said totals and said constraints.

35. The system of claim 34, further comprising a learning engine configured to additionally use past query information from a given user to modify said at least one of a personal menu and a personal exercise program.

36. The system of claim 20, wherein said dieting and exercise model comprises a points-and-constraints-based system in which different kinds and portion sizes of food are assigned points in a first polarity, different kinds, levels and durations of exercise are assigned points in a second, opposite polarity, and constraints are set of at least one of a maximum of exercise points, and points required from different kinds of food, and said advice calculator totals food and exercise to a total determined from a respective personal profile and further calculates both a personal menu and a personal exercise program using said totals and said constraints.

37. An Interactive system for diet and exercise comprising:
a user profile builder for obtaining information from a user and building a profile of data from said user relevant to diet and exercise;
a diet and exercise model for utilizing said profile to assign a given user with a score indicating nutritional need;
a database of nutritional plans associated with different scores;
a database of exercise plans;
a personalization unit for assigning a given user with a combined nutritional and exercise plan;
a scheduler for scheduling notifications to users of their respective plans;
a query unit for receiving queries from respective users and responding with requested data or with modifications to said plans; and
a learning unit for discerning patterns in queries of respective users and using said patterns to modify said nutritional plans to provide a set of customized nutritional plans for said respective users.

38. The system of claim 37, wherein said combined nutritional and exercise plan is in accordance with a user respective score.

39. The system of claim 37, wherein said users are remotely connected thereto by an IP-based network.

40. The system of claim 37, wherein said users are remotely connected thereto via a cellular telephony network.

41. The system of claim 37, wherein said users are remotely connected thereto via an interactive television network.

42. The system of claim 37, wherein said users are remotely connected thereto via the public switched telephone network.

43. A method for interactive support for users for a diet and exercise program comprising:
providing an electronic diet and exercise model,
providing a database of personal profiles of a plurality of remote users,
providing a communication unit for communicating with said plurality of remote users over a network,
using said diet and exercise model together with respective personal profiles to plan a personalized diet and exercise program for a given remote user,
obtaining at least one query from said given remote user,
using said query with said diet and exercise model and said corresponding personal profile to provide a modification to said personalized diet and exercise program, and
sending said modified advice to said remote user.

44. A method for interactive support of users in a diet and exercise program comprising:
providing an electronic diet and exercise model,
constructing a database of personal profiles of a plurality of remote users,
communicating with said plurality of remote users over a network,
using said electronic diet and exercise model together with respective personal profiles to plan a personalized diet and exercise program for a given user, and
scheduling sending of details of said program to said remote user thereby to provide said user with a time-based diet and exercise program.

45. A method for interactive support of users in a diet and exercise program comprising:
obtaining information from a user and building a profile of data from said user relevant to diet and exercise;
utilizing said profile to assign a given user with a score indicating nutritional need;
providing a database of nutritional plans associated with different scores;
providing a database of exercise plans;
assigning a given user with a combined nutritional and exercise plan based on a corresponding score;
scheduling notifications to users of their respective plans;
receiving queries from respective users and responding with requested data or with modifications to said plans; and
discerning patterns in queries of respective users, and
using said patterns to modify said nutritional plans to provide a set of customized nutritional plans for said respective users.
